(19) United States
(12) Patent Application Publication
(10) Pub. No.: US 2025/0197367 A1
(43) Pub. Date: Jun. 19, 2025

(54) PROCESS FOR THE OXIDATION OF HYDROXYMETHYLFURFURAL

(71) Applicant: SÜDZUCKER AG, Mannheim (DE)

(72) Inventors: Sebastian KUNZ, Monsheim (DE); Christine KRÖNER, Kindenheim (DE); Marco WEINMANN, Worms (DE); Alireza HAJI BEGLI, Ramsen (DE)

(21) Appl. No.: 18/838,081

(22) PCT Filed: Feb. 20, 2023
(86) PCT No.: PCT/EP2023/054244

§ 371 Date: Aug. 13, 2024

(30) Foreign Application Priority Data
Feb. 21, 2022 (DE) 102022201794.3

(51) Int. Cl. C07D 307/68

(52) U.S. Cl. CPC C07D 307/68

(57) ABSTRACT

The present invention relates to processes for the preparation of 2,5-furandicarboxylic acid (FDCA) from hydroyxymethylfurfural (HMF) by 2-stage oxidation of the HMF and processes for the preparation of polyesters, polyamides or polyurethanes from the FDCA obtained.

The present invention relates to processes for the preparation of 2,5-furandicarboxylic acid (FDCA) from hydroyxymethylfurfural (HMF) by 2-stage oxidation of the HMF and processes for the preparation of polyesters, polyamides or polyurethanes from the FDCA obtained.

5-Hydroxymethylfurfural (HMF) is a multifunctional molecule with an aromatic 5-ring system, an aldehyde group and an alcohol group. The many functionalities make the molecule a versatile platform chemical that can serve as the basis for a large number of further compounds. The compounds that can be prepared on the basis of HMF include chemicals that are already prepared on an industrial scale by petrochemical means, such as caprolactam or adipic acid, but also compounds with great application potential for which no technical production process with sufficient efficiency is yet available, such as 2,5-furandicarboxylic acid (FDCA). FDCA can serve as a monomer for the preparation of polyethylene furanoate (PEF), which competes with polyethylene terephthalate (PET) as a sustainable raw material base with advantageous technological properties, such as an improved gas barrier effect. Further examples of plastic applications in which FDCA can be used as a terephthalic acid substitute are polybutylene terephthalate (PBT) or polybutylene adipate terephthalate (PBAT). In principle, FDCA can be used in all polymer applications in which diacids are used as monomers.

Formal oxidation of both the aldehyde and the alcohol function of 2,5-hydroxymethylfurfural (HMF) (CAS number 67-47-0) in aqueous solution leads to 2,5-furandicarboxylic acid (FDCA) (CAS number 3238-40-2) via the intermediate products HFCA (hydroxymethyl-2-furancarboxylic acid) (CAS number 6388-41-6) and FFCA (5-formyl-2-furancarboxylic acid) (CAS number 13529-17-4).

Both homogeneous and heterogeneous catalytic oxidation processes for the preparation of FDCA from HMF are known in the prior art. For example, processes are also known which oxidise HMF to FDCA using precious metal catalysts in a one-stage process at basic pH values. Against the background that the complete oxidation of the second aldehyde group of 5-formyl-2-furancarboxylic acid (FFCA), an intermediate product in the oxidation of HMF to FDCA, requires high pH values, it is disadvantageous that HMF is sensitive to high pH values due to its reactivity.

Accordingly, in prior art processes, pH values are often adjusted which represent a compromise between the desired highest possible conversion to FDCA and the pH instability of the HMF to be avoided, but which are therefore not optimally designed for both needs. In addition, common processes for the oxidation of HMF to FDCA generally require long reaction times, which are economically disadvantageous. These processes are usually carried out in a single step, that is the adjustment and maintenance of the desired pH values required for the conversion of HMF under oxygen supply is carried out such that FDCA is formed in a process step without any specific change to the adjusted process parameters, in particular the pH value.

US 2012/0271060 discloses a one-stage process for preparing FDCA from HMF, wherein HMF oxidation is effected at a temperature of more than 140° C. Such a process is neither economical nor gentle due to the temperatures used. EP 2 601 182 A1 discloses a one-stage process for preparing FDCA from HMF, in which weak lyes are used.

The underlying technical problem of the present invention is to overcoming the disadvantages of the prior art and accordingly providing in particular processes which enable an economical oxidation of HMF to FDCA, in particular with a short reaction time, increased FDCA yields and/or better carbon balances.

The present invention solves the underlying technical problem by providing the teachings of the independent and dependent claims and the description.

In particular, the present invention provides a process for the preparation of 2,5-furandicarboxylic acid (FDCA) from hydroxymethylfurfural (HMF) by, in particular 2-stage, oxidation of the HMF, comprising the following process steps:
a) providing an aqueous solution containing HMF having a pH value in a region of 7.0 to 10.0, at least one lye and at least one precious metal catalyst,
b) reacting the aqueous solution containing HMF in the presence of at least one precious metal catalyst provided according to process step a) under oxidative conditions and while maintaining a constant pH value, which is in a region of 7.0 to 10.0, by adding 0.7 to 1.3 amount of substance equivalents (eq) (based on the amount of substance of the HMF in the aqueous solution used) of at least one lye to obtain an intermediate product solution containing hydroxymethyl-2-furancarboxylic acid (HFCA) and/or 5-formyl-2-furancarboxylic acid (FFCA) and
c) reacting the intermediate product solution in a pH value region of 10.5 to 14.0 in the presence of at least one precious metal catalyst provided according to process step a) under oxidative conditions to obtain a solution containing 2,5-furandicarboxylic acid (FDCA).

In a preferred embodiment, the present invention relates to a process as described above, wherein process step c) is carried out while adjusting and maintaining a constant pH value, in particular a single constant pH value, which is in the region of 10.5 to 14.0, by adding the at least one lye.

In particular, the present invention provides a process for preparing 2,5-furandicarboxylic acid (FDCA) from hydroxymethylfurfural (HMF) by, in particular 2-stage, oxidation of the HMF, comprising the following process steps:
a) providing an aqueous solution containing HMF having a pH value in a region of 7.0 to 10.0, at least one lye and at least one precious metal catalyst,
b) reacting the aqueous solution containing HMF in the presence of at least one precious metal catalyst provided according to process step a) under oxidative conditions and while maintaining a constant pH value, which is in a region of 7.0 to 10.0, by adding 0.7 to 1.3 amount of substance equivalents (eq) (based on the amount of substance of the HMF in the aqueous solution used) of at least one lye to obtain an intermediate product solution containing hydroxymethyl-2-furancarboxylic acid (HFCA) and/or 5-formyl-2-furancarboxylic acid (FFCA) and
c) reacting the intermediate product solution in the presence of at least one precious metal catalyst provided according to process step a) under oxidative conditions and while adjusting and maintaining a constant pH value, which is in a region of 10.5 to 14.0, by adding the at least one lye to obtain a solution containing 2,5-furandicarboxylic acid (FDCA).

Accordingly, a process is provided in which FDCA is prepared from HMF by a, preferably two-step, process comprising the process steps (hereinafter also referred to as “steps” for short) a), b) and c). For this purpose, in a process step a), the starting product HMF, at least one lye and at least one precious metal catalyst are provided in an aqueous solution with a pH value in a region of 7.0 to 10.0.

In process step b), that is in a first stage, in the presence of the precious metal catalyst HMF, contained in the aqueous solution, under oxidative conditions while maintaining the pH value provided in process step a), thus, at a constant pH value in a region of 7.0 to 10.0, by addition of 0.7 to 1.3 amount of substance equivalents (eq) of the at least one lye is subsequently reacted, in particular oxidised, to hydroxymethyl-2-furancarboxylic acid (HFCA) and/or 5-formyl-2-furancarboxylic acid (FFCA), wherein the amount of substance equivalents are based on the amount of substance of the HMF in the aqueous solution. An intermediate product solution containing hydroxymethyl-2-furancarboxylic acid (HFCA) and/or 5-formyl-2-furancarboxylic acid (FFCA) with a pH value in a region of 7.0 to 10.0 is obtained.

Over preferably the entire time course of process step b), the amount of substance equivalents of the at least one lye according to the invention is continuously added to the aqueous solution, which is necessary to keep the pH value constant.

Subsequently, that is after the intermediate product solution containing HFCA and/or FFCA has been obtained, wherein preferably all HMF has been reacted, in a process step c), that is in a second stage, the intermediate product solution is further reacted in the presence of the at least one precious metal catalyst under oxidative conditions and while adjusting and maintaining a pH value which, according to the invention, is above the pH value used in the previous process step b) in a region of 10.5 to 14.0, by adding further amounts of the at least one lye. In this process step c), the pH value of the intermediate product solution from process step b) is thus raised to a pH value in a region of 10.5 to 14.0 and then kept constant by adding further at least one lye, wherein HFCA and/or FFCA, in particular both, are reacted, in particular oxidised, to FDCA. In process step c), HFCA and/or FFCA is reacted to FDCA and thus a solution containing FDCA is obtained in this process step.

In process step c), the adding of the at least one lye can preferably be carried out in parts, that is that initially at the beginning of process step c) a part of the at least one lye is added to raise the pH value and then a further part is added continuously to maintain a constant pH value over the entire time course of process step c) to the aqueous intermediate solution.

In a preferred embodiment, the process according to the invention has an additional process step d) following process step c), in which the at least one precious metal catalyst is separated from the solution containing FDCA, in particular by means of filtration.

In a particularly preferred embodiment, the present invention relates to a process according to the invention, wherein in a process step a0) prior to process step a) the pH value of an aqueous solution containing HMF with a pH value of 3.0 to 6.0 is increased to a pH value in a region of 7.0 to 10.0, in particular under non-oxidative conditions, in particular without the addition of air or oxygen.

In a particularly preferred embodiment of the present invention, in process step a0) the pH value can be increased by adding at least one lye.

In a preferred embodiment, in process step a) a lye is provided and is used in both process steps b) and c), in particular in a preferred embodiment also in process step a0).

In a preferred embodiment, in process step a) a strong lye is provided and is used in both process steps b) and c), in particular in a preferred embodiment also in process step a0).

In a particularly preferred embodiment, the at least one lye is NaOH or KOH.

In a preferred embodiment, in process step a) at least two different lyes are provided, wherein these lyes are preferably at least strong lyes.

In a preferred embodiment, in process step a) at least two different lyes are provided, wherein these lyes are preferably each a strong lye and a weak lye.

In a preferred embodiment, in process step a) at least two different lyes are provided, wherein in process step b) the first lye and in process step c) the second lye are used.

In a preferred embodiment, in process step a) at least two different lyes are provided, wherein in process step b) a first, weak lye and in process step c) a second, strong lye are used.

In a particularly preferred embodiment, the first, weak lye, in particular used in process step b), is selected from the group consisting of ammonia, carbonates, hydrogen carbonates, formates and acetates.

In a particularly preferred embodiment, the second, strong lye, in particular used in process step c), is NaOH or KOH.

In a preferred embodiment, the at least one precious metal catalyst provided in process step a) has at least one precious metal, in particular selected from the group consisting of copper, ruthenium, cobalt, iridium, rhodium, platinum, palladium, gold and silver.

In a preferred embodiment, the at least one precious metal catalyst provided in process step a) has at least one precious metal, in particular selected from the group consisting of ruthenium, cobalt, iridium, rhodium, platinum, palladium, gold and silver.

In a particularly preferred embodiment, the precious metal catalyst has at least one precious metal, in particular selected from the group consisting of copper, ruthenium, cobalt, iridium, rhodium, platinum, palladium, gold and silver, and at least one dopant, in particular lead, tin, thallium, tellurium, cobalt or bismuth, in particular bismuth.

In a particularly preferred embodiment, the precious metal catalyst has at least one precious metal, in particular selected from the group consisting of ruthenium, cobalt, iridium, rhodium, platinum, palladium, gold and silver, and at least one dopant, in particular lead, tin, thallium, tellurium, cobalt or bismuth, in particular bismuth.

In a particularly preferred embodiment, the precious metal catalyst is a Pt—Bi precious metal catalyst.

In a particularly preferred embodiment, the precious metal catalyst used in process step b) is a gold catalyst and the precious metal catalyst used in process step c) is a Pt—Bi precious metal catalyst, both catalysts are preferably supported.

In a particularly preferred embodiment, the precious metal catalyst does not contain copper.

In a preferred embodiment, the precious metal catalyst provided in process step a) has at least one support, in particular selected from the group consisting of metal oxides, in particular magnesium oxide, cerium oxide, zirconium oxide, hydroxyapatite, titanium dioxide, hydrotalcite (HAT), Al₂O₃ and carbon (C).

In a particularly preferred embodiment, the amount of precious metal catalyst used, which is provided in process step a), is 0.1 to 25, in particular 1 to 25, in particular 2 to 20, in particular 5 to 15, in particular 7 to 14, in particular 5 g/l (based on the volume of the aqueous solution containing HMF).

In a particularly preferred embodiment, the amount of HMF used, which is provided in process step a), is 0.1 to 2.5, in particular 0.2 to 2.0, in particular 0.3 to 1.5, in particular 0.4 to 0.8, in particular 0.4 mol/l (based on the volume of the aqueous solution containing HMF).

In a preferred embodiment, in process step b) 0.8 to 1.2, in particular 0.9 to 1.1 amount of substance equivalents (eq) (based on the amount of substance of the HMF in the aqueous solution used) are added to the at least one lye.

In a preferred embodiment, in process step c) 0.7 to 1.3, in particular 0.8 to 1.2, in particular 0.9 to 1.1 amount of substance equivalents (eq) (based on the amount of substance of the HMF in the aqueous solution used) are added to the at least one lye.

In a particularly preferred embodiment of the present invention, in process step c) the same at least one precious metal catalyst is used as in the previous process step b), in particular in process step c) the at least one precious metal catalyst used in process step b) is used.

In a particularly preferred embodiment of the present invention, the at least one precious metal catalyst used in process step b) is not separated from the intermediate product solution containing hydroxymethyl-2-furancarboxylic acid (HFCA) and/or 5-formyl-2-furancarboxylic acid (FFCA) and is thus also used in process step c).

In a particularly preferred embodiment of the present invention, in process step c) a different precious metal catalyst is used than in the previous process step b).

In a preferred embodiment, in process step a) two precious metal catalysts are provided, wherein in process step b) a first precious metal catalyst and in process step c) a second precious metal catalyst are used, wherein the first precious metal catalyst is not the same as the second precious metal catalyst.

In a preferred embodiment, in process step a) two precious metal catalysts are provided, wherein in process step b) a first precious metal catalyst and in process step c) a second precious metal catalyst are used, wherein the first precious metal catalyst has a different precious metal than the second precious metal catalyst.

In a preferred embodiment, in process step a) two precious metal catalysts or more precious metal catalysts are provided, wherein in process step b) a first precious metal catalyst and in process step c) another precious metal catalyst are used, wherein the first precious metal catalyst is not the same as the other precious metal catalyst.

In a preferred embodiment, in process step a) two or more precious metal catalysts are provided, wherein in process step b) a first precious metal catalyst and in process step c) another precious metal catalyst are used, wherein the first precious metal catalyst has a different precious metal than the other precious metal catalyst.

In a particularly preferred embodiment, the oxidative conditions, in particular during process steps b) and/or c), are reaction conditions which lead to a supply of oxygen or air into the aqueous solution, in particular by an air or oxygen gassing device, in particular a gassing stirrer or a blower or/and a flow channel, in particular with simultaneous mechanical agitation of the solution, for example by an agitator or a turbulence means.

In a particularly preferred embodiment, the oxidative conditions, in particular during process steps b) and/or c), in particular b) and c), are the supply of oxygen into the solution by an air or oxygen gassing device, in particular by an air or oxygen gassing device with a flow rate of 10 to 5000, in particular 100 to 5000, in particular 400 to 2000, in particular 600 to 1500, in particular 1000 ml/min per litre of reaction volume.

In a particularly preferred embodiment, the oxidative conditions, in particular during process steps b) and/or c), in particular b) and c), are the supply of air into the solution by an air or oxygen gassing device, in particular by an air or oxygen gassing device with a flow rate of 50 to 25000, in particular 500 to 25000, in particular 2000 to 10000, in particular 3000 to 7500, in particular 5000 ml/min per litre of reaction volume.

In a particularly preferred embodiment, the oxidative conditions, in particular during process steps b) and/or c), in particular b) and c), are the supply of oxygen into the solution by an air or oxygen gassing device, in particular with a flow rate of 10 to 5000, in particular 100 to 5000, in particular 400 to 2000, in particular 600 to 1500, in particular 1000 ml/min per litre of reaction volume, and a mechanical agitation of the solution, for example by an agitator or a turbulence means.

In a particularly preferred embodiment, the oxidative conditions, in particular during process steps b) and/or c), in particular b) and c), are the supply of air into the solution by an air or oxygen gassing device, in particular with a flow rate of 50 to 25000, in particular 500 to 25000, in particular 2000 to 10000, in particular 3000 to 7500, in particular 5000 ml/min of air per litre of reaction volume, and a mechanical agitation of the solution, for example by an agitator or a turbulence means.

In a particularly preferred embodiment, the oxidative conditions in process steps b), c) or b) and c) are provided with the aid of a gassing stirrer.

A gassing agitator preferably used according to the invention in steps b) and c) provides both the functions of an air or oxygen gassing device and an agitator.

In a preferred embodiment, process steps b) and/or c) are carried out with mechanical agitation, in particular with stirring or swirling, in particular by means of a gassing agitator.

In a preferred embodiment, in process step b) the aqueous solution is stirred by means of the agitator, in particular at a speed of 50 to 5000, in particular 200 to 1000, in particular 300 to 900, in particular 400 to 500 rpm (revolutions of the agitator shaft per minute, also referred to as rpm).

In a preferred embodiment, in process step c) the aqueous solution is stirred by means of the agitator, in particular at a speed of 50 to 5000, in particular 200 to 1000, in particular 300 to 900, in particular 400 to 500 rpm (revolution of the agitator shaft per minute).

In a preferred embodiment, in process steps b) and c) the aqueous solution is stirred by means of the agitator, in particular at a speed of 50 to 5000, in particular 200 to 1000, in particular 300 to 900, in particular 400 to 500 rpm (revolution of the stirrer shaft per minute).

In a particularly preferred embodiment, process steps a0), a), b) and c), in particular a), b) and c), in particular b) and c), are carried out at a pressure of normal pressure to 20 bar, in particular normal pressure to 10 bar, in particular normal pressure to 5 bar.

In a particularly preferred embodiment, process steps a0), a), b) and c), in particular a), b) and c), in particular b) and c) are carried out at normal pressure.

In a preferred embodiment, process steps b) or c) or both process steps are carried out at a temperature of 40 to 100° C., in particular at a temperature of 50 to 90° C., in particular at a temperature of 55 to 80° C., in particular of 55 to 65° C., in particular at 60° C.

In a preferred embodiment, the process according to the invention has, following process step c) or d), an additional process step f) in which the FDCA obtained in process step c), preferably as anion, that is non-protonated FDCA, is protonated, in particular by adjusting the solution containing FDCA to a pH value of 1.0 to 6.5 and precipitating FDCA.

In a preferred embodiment, the process according to the invention has, following process step c) or d), an additional process step f) in which the solution containing FDCA is adjusted to a pH value of 1.0 to 6.5, FDCA is precipitated and protonated FDCA is obtained.

In a preferred embodiment, the process according to the invention has, following process step c) or d), and preferably prior to process step f), in particular following process step d) and prior to process step f), an additional process step e) in which the solution containing FDCA is purified.

In a preferred embodiment, the process according to the invention has, following process step c) or d) or f), in particular following d) or f), an additional process step g) in which the protonated (after process step f)) or non-protonated FDCA (after process step c) or d)) is purified, preferably by washing, recrystallisation, melting, adsorption or a combination thereof.

In a preferred embodiment, the process according to the invention has, following process step f), an additional process step g) in which protonated or non-protonated FDCA is purified, preferably by washing, recrystallisation, melting, adsorption or a combination thereof.

In a preferred embodiment, according to process step g) isolated FDCA is obtained.

In a preferred embodiment, the process according to the invention has, following process step c), d), e), f) or g), an additional process step h) in which the protonated or non-protonated FDCA is converted into a methyl, ethyl or dimethyl ester.

The present invention also relates to processes for the preparation of polyesters, polyamides or polyurethanes, in particular polyethylene furanoate (PEF), wherein said process comprises a process according to the invention for the preparation of FDCA and subsequently polymerising the FDCA obtained from process step c), d), e), f) or g), preferably following process step d), e), f) or g).

In a preferred embodiment, the process for the preparation of polyesters, polyamides or polyurethanes comprises purifying the FDCA obtained from process step c), d) or f), preferably d) or f), prior to the polymerising, in particular according to process step g).

In a particularly preferred embodiment, the process according to the invention consists of process steps a), b), and c), in particular no further process steps are carried out between these process steps.

In a preferred embodiment, between process steps b) and c) no further process steps are carried out.

In a preferred embodiment, between process steps b) and c) no purification step is carried out.

In a preferred embodiment, the intermediate product solution obtained in process step b) containing hydroxymethyl-2-furancarboxylic acid (HFCA) and/or 5-formyl-2-furancarboxylic acid (FFCA) is reacted in process step c) without any further process steps, in particular purification steps and/or separation steps, in particular filtration steps.

In a preferred embodiment, the process has, following process step b) and prior to process step c), a process step b1), in which the at least one precious metal catalyst is separated from the intermediate product solution containing hydroxymethyl-2-furancarboxylic acid (HFCA) and/or 5-formyl-2-furancarboxylic acid (FFCA), in particular by filtration.

In a particularly preferred embodiment, the process has, following process step b1) and prior to process step c), a process step b2), in which the at least one precious metal catalyst, in particular the at least one precious metal catalyst not equal to the at least one precious metal catalyst separated off in process step b1), is added to the intermediate product solution containing hydroxymethyl-2-furancarboxylic acid (HFCA) and/or 5-formyl-2-furancarboxylic acid (FFCA).

In the context of the present invention, a “separation” or a “separation step” is understood to mean a separating of at least one catalyst from a solution, in particular intermediate product solution containing hydroxymethyl-2-furancarboxylic acid (HFCA) and/or 5-formyl-2-furancarboxylic acid (FFCA) or solution containing 2,5-furandicarboxylic acid (FDCA), wherein the composition of the solution, in particular intermediate solution containing hydroxymethyl-2-furancarboxylic acid (HFCA) and/or 5-formyl-2-furancarboxylic acid (FFCA) or solution containing 2,5-furandicarboxylic acid (FDCA), remains unchanged. Preferably, after the separation or after the separation step, the compounds contained in the solution, in particular intermediate product solution containing hydroxymethyl-2-furancarboxylic acid (HFCA) and/or 5-formyl-2-furancarboxylic acid (FFCA) or solution containing 2,5-furandicarboxylic acid (FDCA), in particular educts, products and by-products, are present in the same ratio, in particular volume and/or amount of substance ratio, to one another as prior to the separation or prior to the separation step.

In the context of the present invention, a “separation” or a “separation step of at least one catalyst from a solution, in particular an intermediate product solution containing hydroxymethyl-2-furancarboxylic acid (HFCA) and/or 5-formyl-2-furancarboxylic acid (FFCA) or solution containing 2,5-furandicarboxylic acid (FDCA), is not a purification or purification step of a compound, in particular intermediate product or product, in particular hydroxymethyl-2-furancarboxylic acid (HFCA) and/or 5-formyl-2-furancarboxylic acid (FFCA) or 2,5-furandicarboxylic acid (FDCA) contained in a solution, in particular an intermediate solution containing hydroxymethyl-2-furancarboxylic acid (HFCA) and/or 5-formyl-2-furancarboxylic acid (FFCA) or solution containing 2, 5-furandicarboxylic acid (FDCA).

In the context of the present invention, “purification” or “purification step” is understood to mean a removal of educts and/or by-products from a mixture containing a product and the educts and/or by-products. Preferably, purification or purification step is understood to mean a removal of educts, in particular HMF, intermediate products, in particular hydroxymethyl-2-furancarboxylic acid (HFCA) and/or 5-formyl-2-furancarboxylic acid (FFCA), and/or by-products of 2,5-furandicarboxylic acid (FDCA). Preferably, the purification or purification step is a washing, a recrystallisation, a melting, an adsorption, an extraction, a distillation or a combination thereof.

In a particularly preferred embodiment of the present invention, the process according to the invention comprises process steps a), b) and c), wherein no further process steps are carried out between process steps a), b) and c), but optionally prior to process step a) and/or following process step c) further process steps are carried out, in particular process step a0) prior to process step a) and at least one of process steps d) to g) following process step c).

According to the invention, the present process comprises process steps a) to c), preferably a0) to c), in particular a) to d), preferably a0) to d), preferably a) to e), preferably a0) to e), in particular a) to f), preferably a0) to f), preferably a) to g), preferably a0) to g). According to the invention, the present process particularly preferably comprises the process steps a0), a), b), c), d), e) and f). However, it may also be provided that the present process comprises steps a), b), c), d), e) and f). Particularly preferred according to the invention, the present process comprises the process steps a0), a), b), c), d), e), f) and g). In a particularly preferred embodiment according to the invention, the present process comprises process steps a), b), c), d), e), f) and g).

In a particularly preferred embodiment, the present process consists of process steps a) to c), preferably a) to d), preferably a) to e), preferably a) to f), preferably a0) to c), in particular a0) to d), in particular a0) to e), in particular a0) to f), preferably a0) to g), preferably a) to g).

In a preferred embodiment, the process is carried out in the sequence of process steps a), b), c) and, if optionally provided, d), e) and f), in particular a0), a), b), c) and, if optionally provided, d), e), f) and g).

According to the invention, in a preferred embodiment in the process for the preparation of FDCA according to at least process steps a) to c), the reacting of HMF to FDCA is carried out in a batch process, that is with batchwise feed of educts and withdrawal of products, preferably in a batch reactor system, in particular a reactor.

According to the invention, in one embodiment in the process for preparation of FDCA according to at least steps a) to c), the reacting of HMF to FDCA is carried out in a continuous process, that is with constant feed of educts and withdrawal of products, preferably in a continuous reactor system.

According to the present invention, neither HMF nor the intermediate products and FDCA are used, reacted or prepared during the process in a solvent other than water, in particular not in an organic solvent. The process according to the invention is preferably free from the use of organic solvents.

In a preferred embodiment, water is used as the sole solvent in the process according to the invention.

The present invention therefore provides in particular a process for the preparation 2,5-furandicarboxylic acid (FDCA) from hydroxymethylfurfural (HMF) by, in particular 2-stage, oxidation of the HMF, comprising the following process steps:
a) providing an aqueous solution containing HMF having a pH value in a region of 7.0 to 10.0, at least one lye and at least one precious metal catalyst,
b) reacting the aqueous solution containing HMF in the presence of the at least one precious metal catalyst under oxidative conditions and while maintaining a constant pH value, which is in a region of 7.0 to 10.0, by adding 0.7 to 1.3 amount of substance equivalents (eq) (based on the amount of substance of the HMF in the aqueous solution used) of the at least one lye to obtain an intermediate product solution containing hydroxymethyl-2-furancarboxylic acid (HFCA) and/or 5-formyl-2-furancarboxylic acid (FFCA) and
c) reacting the intermediate solution in the presence of the at least one precious metal catalyst under oxidative conditions and while adjusting and maintaining a constant pH value, which is in a region of 10.5 to 14.0, by adding the at least one lye to obtain a solution containing 2,5-furandicarboxylic acid (FDCA).

In the context of the present invention, normal pressure is understood to be the gas pressure corresponding to an average air pressure at the earth's surface of 101 325 Pa=1.013 25 bar.

In the context of the present invention, an “agitator” is understood to mean an agitating device, that is a device which has an agitating element arranged on at least one agitator shaft and is configured to stirr a solution.

In the context of the present invention, a “turbulence means” is understood to be a device, which is configured to cause flow movements in a solution.

In the context of the present invention, a “weak lye” is understood to mean a lye having a pKS of 10.4 to 3.75.

In the context of the present invention, a “strong lye” is understood to mean a lye with a pKb of <3.

In the context of the present invention, the term “amount of substance equivalents (eq)” is understood to mean the amount of substance in mol, which corresponds to the amount of substance to which reference is made. For example, if 3 mol of compound A (concentration A 1.5 mol/L) are present in a solution with a volume of 2 L and 0.5 amount of substance equivalents of compound B are to be added, these 0.5 eq therefore correspond to 1.5 mol to be added to the solution (concentration B 0.75 mol/L).

In the context of the present invention, “oxidative conditions” are understood to mean reaction conditions or reaction environments which are characterised in particular by the process parameters temperature, pH, pressure and presence of oxygen such that oxidation of a starting compound, in particular HMF, or intermediate product, in particular HFCA and/or FFCA, is enabled, in particular during process steps b) and/or c). In the context of the present invention, “oxidative conditions” are understood to mean in particular reaction conditions which are characterised by a supply of oxygen or air into the aqueous solution, for example by an air or oxygen gassing device, such as a gassing stirrer or a blower or/and a flow channel, in particular with simultaneous mechanical agitation of the solution, for example by an agitator or a turbulence means.

In the context of the present invention, “non-oxidative conditions” are understood to mean reaction conditions or reaction environments which are characterised in particular by the process parameters temperature, pH, pressure and presence of oxygen such that oxidation of a starting material, in particular HMF or intermediate product, in particular HFCA and/or FFCA, is not enabled, in particular during process steps b) and/or c). In the context of the present invention, “non-oxidative conditions” are understood to mean, in particular, reaction conditions which are not characterised by a supply of oxygen or air into the aqueous solution.

In the context of the present invention, a “supply” of air or oxygen is understood to mean an influx, that is local increase in concentration, of air or oxygen into the aqueous solution which is specifically brought about by suitable process measures and in particular by using air or oxygen gassing devices, in particular a gassing stirrer, which goes beyond pure diffusion processes. This supply is preferably a continuous supply. The supply serves to compensate for the oxygen present in the aqueous solution which is consumed by the reaction, so that there is no depletion of oxygen in the aqueous solution.

In the context of the present invention, “air” is understood to mean a gas mixture which has a volume fraction of oxygen of 20 to 22, in particular about 21 volume-%, in particular 21 volume-% (based on the total volume of air).

In the context of the present invention, a “2-stage” or “two-stage” process is understood to mean a process which is characterised by an initial state in which the at least one desired chemical reaction starts and the initial reaction conditions are or are preferably adjusted, and a final state in which the desired, in particular maximum, amount of starting material has been reacted, wherein between the initial and final states the reaction conditions, in particular the pH value, have been specifically changed at least once by an operator, human or artificial. In comparison, a one-stage process is characterised in that the pH value has not been specifically changed by an operator between the initial and final states, in particular no reaction conditions have been changed at all.

In the context of the present invention, the term “constant” pH value or “maintaining a constant pH value” is understood to mean that the specific pH value mentioned in each case is to be kept constant with a maximum deviation of +/−0.2 pH value units, in particular that the specific pH value mentioned in each case is to be kept constant with a maximum deviation of +/−0.1 pH value units, in particular has exactly the specified pH value.

In the context of the present invention, the pH value is determined as follows:

For example, a titrator, for example Dulcometer PHD from company Prominent, is used for pH value adjustment and control, which combines pH value measurement and lye dosing. For example, a pH electrode from Mettler Toledo HA 405-DXK-S8/325 or a comparable electrode from another manufacturer is used as the pH electrode. The pH electrode is calibrated with 2 buffer solutions (for example WTW technical buffers) with pH 7.00 and pH 10.01 at 25° C. prior to use. A Pt-100 element is connected to the titrator for temperature compensation, as the reaction is carried out at temperatures higher than 25° C.

In the context of the present invention, “protonated FDCA” is understood to mean 2,5-furandicarboxylic acid, CAS number 3238-40-2.

In the context of the present invention, “non-protonated FDCA” is understood to mean a monovalent or divalent anion of 2,5-furandicarboxylic acid.

In the context of the present invention, “FDCA” is understood to mean 2,5-furandicarboxylic acid, CAS number 3238-40-2, which, if present in solution, may be present in protonated or non-protonated form depending on the pH value of the solution containing FDCA.

In the context of the present invention, the term “at least one” is understood to mean a quantity expressing a number of 1 or 2 or 3 or 4 or 5 or 6 or 7 or 8 or 9 or 10 and so on. In a particularly preferred embodiment, the term “at least one” may represent exactly the number 1. In a further preferred embodiment, the term “at least one” may also mean 2 or 3 or 4 or 5 or 6 or 7.

In the context of the present invention, the term “at least one precious metal catalyst” is understood to mean a numerically defined number of precious metal catalysts, wherein the number may be 1, 2, 3, 4, 5, 6, 7, 8, 9, 10 or more.

Where quantitative data, in particular percentages, of components of a product or a composition are given in the context of the present invention, these add up to 100% of the composition and/or the product together with the other explicitly stated or evident to a person skilled in the art further components of the composition or the product, unless explicitly stated otherwise or evident to a person skilled in the art.

Where, in the context of the present invention, a “presence”, a “containment”, a “presence” or a “containing” of a component is explicitly mentioned or implied, this means that each component is present, in particular is present in a measurable amount.

The number of decimal places indicated corresponds to the precision of the measurement method used in each case.

If, in the context of the present invention, the first and second decimal places or the second decimal place are/is not specified, these are/is to be set as 0.

In the context of the present invention, the term “and/or” is understood to mean that all members of a group which are connected by the term “and/or” are disclosed both alternatively to each other and cumulatively to each other in any combination. This means for the expression “A, B and/or C” that the following disclosure content is to be understood: a) A or B or C or b) (A and B), or c) (A and C), or d) (B and C), or e) (A and B and C).

In the context of the present invention, the terms “comprising” and “having” are understood to mean that additionally to the elements explicitly covered by these terms, further elements not explicitly mentioned may be added. In the context of the present invention, these terms are also understood to mean that only the explicitly mentioned elements are included and that no further elements are present. In this particular embodiment, the meaning of the terms “comprising” and “having” is synonymous with the term “consisting of”. Furthermore, the terms “comprising” and “having” also encompass compositions which, in addition to the explicitly mentioned elements, also contain further elements which are not mentioned but which are of a functional and qualitatively subordinate nature. In this embodiment, the terms “comprising” and “having” are synonymous with the term “consisting essentially of”. The term “consisting of” means that only the explicitly mentioned elements are present and the presence of further elements is excluded.

Further embodiments of the present invention are the objects of the subclaims and further independent claims.

The invention is explained in more detail with reference to the following examples and the associated figures.

The figures show:

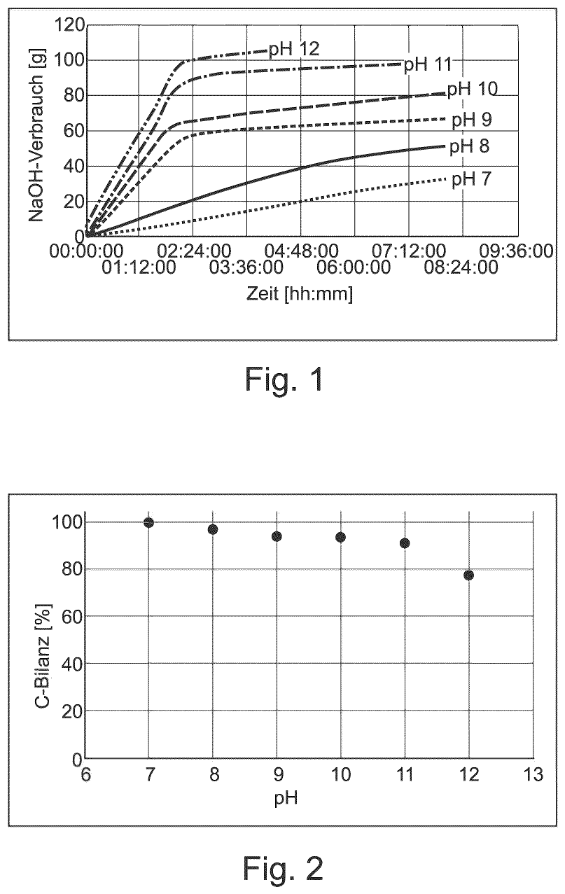

FIG. 1: NaOH consumption curves as a function of the pH value set in process step b) for experiments 5a-f.

FIG. 2: C-balance as a function of the pH value set in process step b) for experiments 5a-f.

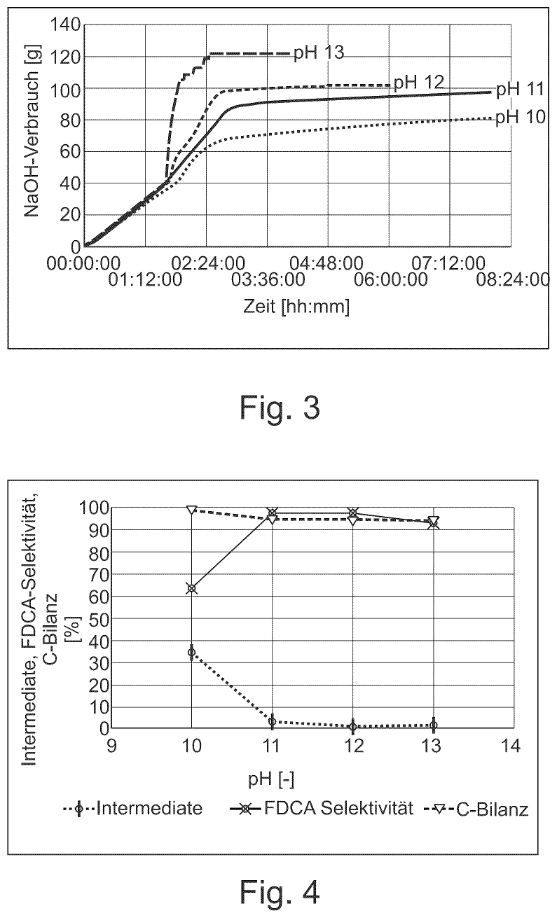

FIG. 3: NaOH consumption curves as a function of the pH value set in process step c) for experiments 6a-6d.

FIG. 4: Selectivity for intermediates and HMF as well as C-balance as a function of the pH value set in process step c) for experiments 6a-6d.

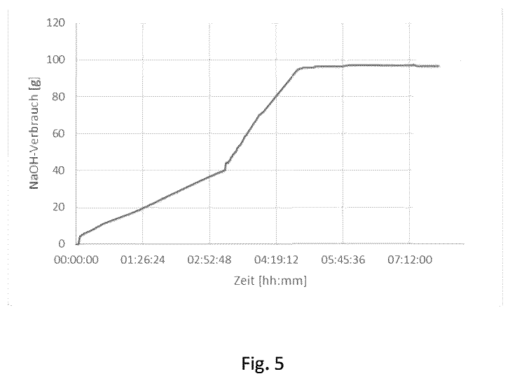

FIG. 5: NaOH consumption curve for the process according to the invention using two different catalysts.

### EXAMPLES

### Example 1—One-Stage Process (Comparative Process)

An activated carbon-supported platinum/bismuth catalyst (3.5% Pt/1% Bi/C(=Norit SX UltraCat)) was used as a catalyst. The HMF oxidation was carried out under the following reaction conditions:
Reaction volume (batch): 500 ml
Catalyst amount: 10.1 g/l
Initial substrate concentration: 0.4 mol/l
pH: 9.0
Temperature: 60° C.
Pressure: Normal pressure
O₂ flow rate: 0.5 l/min
Titrant: NaOH (16 wt. % dry matter (DM) to total mass)
Stirring speed: 300 rpm (agitator)

The reaction time was once 5.8 h (example 1a) and once 23.1 h (example 1b) with otherwise identical reaction conditions.

### Example 2—Two-Stage Process According to the Invention

The same catalyst was used as in example 1 (3.5% Pt/1% Bi/C(=Norit SX UltraCat)). The HMF oxidation was carried out under the following reaction conditions:
Reaction volume: 500 ml
Catalyst amount: 9.5 g/l
Initial substrate concentration: 0.4 mol/l
pH, steps a) and b): 9.0
pH, step c) 12.0
Temperature: 60° C.
Pressure, steps b) and c): Normal pressure
O₂ flow rate, steps b) and c): 0.5 l/min
Titrant, steps b) and c): NaOH (16 wt. % dry matter (DM) to total mass)
Stirring speed, steps b) and c): 300 rpm (agitator)

The changeover to process step c) took place after the addition of 1.13 equivalents of NaOH in process step b), during which the pH value was kept constant.

To adjust and maintain the pH value in process step c), 0.88 equivalents of NaOH were added.

The results obtained under the reaction conditions from examples 1 and 2 are presented in Table 1.

TABLE 1
Comparison of the results from examples 1 and 2.

| | Example 1a - one- | Example 1b - one- | Example 2 -2-stage |
|---|---|---|---|
| | stage comparative | stage comparative | according to the |
| Process | process | process | invention |
| Reaction time | 5.8 h | 23.1 h | 0.8 h |
| C-balance at the end | 85.6 | 87.7 | 92.3 |
| of the reaction | | | |
| FDCA [g/100 g] | 2.19 | 4.65 | 4.62 |
| FFCA [g/100 g] | 1.96 | 0.4 | 0.08 |
| HFCA [g/100 g] | 0.06 | 0.02 | 0.02 |

It can be seen that in example 2 according to the invention the C-balance is significantly better than in comparative example 1. Furthermore, in example 2 a comparable amount of FDCA could be prepared in a considerably shorter time than in example 2b, wherein the content of minor components is lower.

### Example 3—Addition of 2 and 4 Lye Equivalents at the Start of the Reaction (Comparative Example)

A hydrotalcite-supported Pt/bismuth catalyst (2.5% Pt/1% Bi/HT) was used as a catalyst. The HMF oxidation was carried out under the following reaction conditions:
Reaction volume (batch): 500 ml
Catalyst amount: 5.0 g/l
Initial substrate concentration: 0.4 mol/l
pH: no constant pH value, pH start 13.2-pH end 9.6
Temperature: 60° C.
Pressure: normal pressure
O₂ flow rate: 0.5 l/min
Titrant: NaOH (16 wt. % dry matter (DM) to total mass)
Stirring speed: 300 rpm (agitator)

### Example 3a

The total amount of NaOH required of 100 g NaOH (16 wt %) corresponding to 2 equivalents of NaOH per mole of HMF was added directly at the start of the reaction.

### Example 3b

The total amount of NaOH required of 200 g NaOH (16 wt %) corresponding to 4 equivalents of NaOH per mole of HMF was added directly at the start of the reaction.

### Example 4—Process According to the Invention

The same catalyst was used as in example 3. The HMF oxidation was carried out under the following reaction conditions:
Reaction volume (batch): 500 ml
Catalyst amount: 5.0 g/l
Initial substrate concentration: 0.4 mol/l
pH, steps a) and b): 9.0
pH, step c): 11.6
Temperature, steps b) and c): 60° C.
Pressure, steps b) and c): Normal pressure
O₂ flow rate, steps b) and c): 0.5 l/min
Titrant, steps b) and c): NaOH (16 wt. % dry matter (DM) to total mass)
Stirring speed, steps b) and c): 300 rpm (agitator)

The changeover to process step c) took place after the addition of 0.8 equivalents of NaOH in process step b), during which the pH value was kept constant. To adjust and maintain the pH value in process step c), 1.2 equivalents of NaOH were added. A total of 2 NaOH equivalents were added.

The results from examples 3a, 3b and 4 are presented in Table 2.

TABLE 2
Comparison of the test results from examples 3 and 4.

| | Example 3a - 2 | Example 3b - 4 | Example 4 -2-stage |
|---|---|---|---|
| | equivalents of NaOH | equivalents NaOH | according to the |
| Process | direct addition | direct addition | invention |
| Reaction time | 5 h | 5 h | 4.82 h |
| C-balance at the end | 11.31 | 21.07 | 95.14 |
| of the reaction | | | |
| FDCA [g/100 g] | 0.00 | 0.84 | 4.81 |
| FFCA [g/100 g] | 0.29 | 0.01 | 0.06 |
| HFCA [g/100 g] | 0.26 | 0.11 | 0.00 |

It can be seen that in example 4 according to the invention, a significantly better C-balance, more FDCA and fewer intermediate products were obtained than in the two comparative examples 3a and 3b.

### Example 5—Variation of the pH Value in Process Step b)

A hydrotalcite-supported platinum/Bi catalyst (2.5% Pt/1% Bi/HT) was used as a catalyst.

HMF oxidation was carried out under the following reaction conditions in process steps a) and b):
Reaction volume (batch): 500 ml
Catalyst amount: 5.0 g/l
Initial substrate concentration: 0.4 mol/l
Temperature: 60° C.
Pressure: normal pressure
O₂ flow rate: 0.5 l/min
Titrant: NaOH 16 (16 wt. % dry matter (DM) to total mass)
Stirring speed: 900 rpm (Büchi gassing stirrer)

The pH values in process step b) were selected as follows (Table 3) and kept constant by adding appropriate amounts of NaOH. Process step c) was not carried out.

TABLE 3
pH values in process step b) for experiments 5a-f.

| | | Equivalents of |
|---|---|---|
| | pH value, step | NaOH added in |
| No. | b) | step b) |
| 5a | 7.0 | 0.6 |
| 5b | 8.0 | 1.0 |
| 5c | 9.0 | 1.3 |
| 5d | 10.0 | 1.6 |
| 5e | 11.0 | 1.9 |
| 5f | 12.0 | 2.1 |

In FIG. 1 a comparison of the titration curves is shown.

It is clearly visible that the speed of the reacting in process step b) is dependent on the pH value.

As the pH value increased, the reaction became faster. In FIG. 2 the C-balance at the end point of process step b) (addition of 1 equivalent of NaOH (equivalent to 50 g NaOH) is shown.

The higher reaction speed at higher pH values is accompanied by an increasing loss of product and/or intermediate products. Acceptable C-balances of >93% are only achieved for pH values ≤pH 10.

### Example 6—Variation of the pH Value in Process Step c)

A hydrotalcite-supported platinum/Bi catalyst (2.5% Pt/1% Bi/HT) was used as the catalyst.

The HMF oxidation was carried out under the following reaction conditions:
Reaction volume (batch): 500 ml
Catalyst amount: 5.0 g/l
Initial substrate concentration: 0.4 mol/l
pH value, steps a) and b): 9.0
Temperature, steps b) and c): 60° C.
Pressure, steps b) and c): Normal pressure
O₂ flow rate, steps b) and c): 0.5 l/min
Stirring speed, steps b) and c): 900 rpm (Büchi gassing stirrer) The changeover to process step c) took place after the addition of 0.8 equivalents of NaOH in process step b).

The pH values for process step c) were selected for the different examples as shown in Table 4 and adjusted and kept constant by adding appropriate amounts of NaOH:

TABLE 4
pH values in process step c) for experiments 6a-d.

| | | Equivalents of |
|---|---|---|
| | pH value, step | NaOH added in |
| No. | c) | step c) |
| 6a | 7.0 | 0.8 |
| 6b | 8.0 | 1.2 |
| 6c | 9.0 | 1.2 |
| 6d | 10.0 | 1.6 |

In FIG. 3 the titration curves for the different pH values of process step c) are shown.

In FIG. 4 the C-balance, the FDCA selectivity and the selectivity for the intermediates as a function of the pH value are shown.

The C-balance is highest for pH 10 at 98%, but the selectivity for FDCA is also lowest at 64%. The high selectivity for the intermediates (34%) shows that the reaction was not yet complete. The highest selectivity for FDCA is 97.5% at a pH of 12. Under these conditions, the selectivity for the intermediates is also the lowest at 0.44%.

### Example 7—Use of Different Catalysts for Stages 1 and 2

Catalyst 1st stage: 1% Au/Al₂O₃
Catalyst 2nd stage: 2.5% Pt/1.0% Bi/HAT (HT=hydrotalcite)

The reaction was carried out under the following reaction conditions:
Reaction volume: 500 ml
Catalyst amount 1st stage: 12.5 g/l
Catalyst amount 2nd stage: 5.0 g/l
Initial substrate concentration: 0.4 mol/l
pH 1st stage: 9.0
pH 2nd stage: 11.7
Temperature: 60° C.
Pressure: Normal pressure
O₂ flow rate: 500 ml/min
Stirring speed: 900 rpm (Büchi gassing stirrer)

After adding 0.8 equivalents of NaOH in process step b), the catalyst is separated by filtration and the filtered solution is used without further treatment in the second stage in process step c) with the addition of 1.1 equivalents of NaOH.

FIG. 5 shows the NaOH consumption curve for the process according to the invention using two different catalysts.

Table 5 shows the results.

| | |
|---|---|
| HMF-conversion | 100% |
| C-balance | 97.03% |
| FDCA-selectivity | 96.83% |
| Intermediate | 0.21% |

Complete HMF conversion is achieved with acceptable C-balance and high FDCA selectivity.

1. A process for the preparation of 2,5-furandicarboxylic acid (FDCA) from hydroxymethylfurfural (HMF) by 2-stage oxidation of the HMF, comprising the following process steps:
a) providing an aqueous solution containing HMF having a pH value in a region of 7.0 to 10.0, at least one lye and at least one precious metal catalyst,
b) reacting the aqueous solution containing HMF in the presence of at least one precious metal catalyst provided according to process step a) under oxidative conditions and while maintaining a constant pH value, which is in a region of 7.0 to 10.0, by adding 0.7 to 1.3 amount of substance equivalents (eq) (based on the amount of substance of the HMF in the aqueous solution used) of the at least one lye to obtain an intermediate product solution containing hydroxymethyl-2-furancarboxylic acid (HFCA) and/or 5-formyl-2-furancarboxylic acid (FFCA) and
c) reacting the intermediate product solution in a pH value region of 10.5 to 14.0 in the presence of at least one precious metal catalyst provided according to process step a) under oxidative conditions to obtain a solution containing 2,5-furandicarboxylic acid (FDCA).

2. The process of claim 1, wherein process step c) is carried out while adjusting and maintaining a constant pH value, which is in the region of 10.5 to 14.0, by adding the at least one lye.

3. The process of claim 1, wherein in a process step a0) prior to process step a) the pH value of an aqueous solution containing HMF with a pH value of 3.0 to 6.0 is increased to a pH value in a region of 7.0 to 10.0, in particular under non-oxidative conditions, in particular without the addition of air and oxygen.

4. The process of claim 1, wherein in process step a) at least two different lyes are provided and in process step b) a first lye, in particular a weak lye, and in process step c) a second lye, in particular a strong lye, are used.

5. The process of claim 1, wherein the at least one, in particular strong, lye is NaOH or KOH.

6. The process of claim 1, wherein in process step c) 0.7 to 1.3 amount of substance equivalents (eq) of the at least one lye (based on the amount of substance of the HMF in the aqueous solution used) are added.

7. The process of claim 1, wherein the oxidative conditions, in particular during the process steps b) and/or c), are reaction conditions which lead to a supply of oxygen or air into the aqueous solution, in particular by an air or oxygen gassing device, in particular a gassing stirrer or a blower or/and a flow channel, in particular with simultaneous mechanical agitation of the solution, for example by an agitator or a turbulence means.

8. The process of claim 1, wherein the process steps b) and c) are carried out with mechanical agitation, in particular with stirring by means of an agitator or a turbulence means.

9. The process of claim 1, wherein the at least one precious metal catalyst has at least one precious metal selected from the group consisting of copper, ruthenium, rhodium, cobalt, iridium, platinum, palladium, gold and silver.

10. The process of claim 1, wherein the at least one precious metal catalyst has at least one precious metal and at least one dopant, in particular lead, tin, thallium, tellurium, cobalt, or bismuth, in particular is a Pt—Bi precious metal catalyst.

11. The process of claim 1, wherein process step b), c) or both is carried out at a temperature of 40 to 100° C.

12. The process of claim 1, wherein, following process step c), in a process step d) the at least one precious metal catalyst is separated from the solution containing FDCA, in particular by means of filtration.

13. The process of claim 1, wherein, following process step c) or d), in a process step f) the FDCA obtained in process step c) or d) is protonated, in particular by adjusting the solution containing FDCA to a pH value of 1.0 to 6.5 and obtaining protonated FDCA.

14. The process of claim 1, wherein, following process step c) or d), in a process step e) the solution containing the FDCA is purified.

15. The process of claim 1, wherein, following process step c), d) or f), in a process step g) the protonated or non-protonated FDCA is purified, preferably by washing, recrystallisation, melting, adsorption or a combination thereof.

16. The process of claim 1, wherein, following process step c), d), e), f) or g), in a process step h) protonated or non-protonated FDCA is converted into a methyl, ethyl or dimethyl ester.

17. A process for the preparation of polyesters, polyamides or polyurethanes, in particular polyethylene furanoate (PEF), wherein in a process of claim 1 and subsequently a polymerising of the FDCA obtained from process step c), d), e), f) or g) is carried out.
